(19) United States
(12) Patent Application Publication
(10) Pub. No.: US 2024/0172970 A1
(43) Pub. Date: May 30, 2024

(54) APPARATUS AND METHOD FOR ESTIMATING ANTIOXIDANT COMPONENT

(71) Applicant: SAMSUNG ELECTRONICS CO., LTD., Suwon-si (KR)

(72) Inventors: Yoon Jae KIM, Suwon-si (KR); Jin Young PARK, Suwon-si (KR); Hyun Seok MOON, Suwon-si (KR); Kun Sun EOM, Suwon-si (KR); Myoung Hoon JUNG, Suwon-si (KR); Jeong Eun HWANG, Suwon-si (KR)

(73) Assignee: SAMSUNG ELECTRONICS CO., LTD., Suwon-si (KR)

(21) Appl. No.: 18/130,675

(22) Filed: Apr. 4, 2023

(30) Foreign Application Priority Data
Nov. 30, 2022 (KR) 10-2022-0164218

(51) Int. Cl. A61B 5/145; A61B 5/00; A61B 5/1455; G06N 20/00

(52) U.S. Cl. CPC A61B 5/14546; A61B 5/1455; A61B 5/7267; G06N 20/00; A61B 5/441

(57) ABSTRACT

Provided is an apparatus configured to estimate an antioxidant component, the apparatus including a storage configured to store first antioxidant concentrations estimated at a first body part and second antioxidant concentrations estimated at a second body part, and a processor configured to estimate the first antioxidant concentrations at the first body part, to extract, as training data, data pairs of the first antioxidant concentrations and the second antioxidant concentrations from the storage based on learning conditions, and generate a transformation model configured to transfer the second antioxidant concentrations into a reference index based on the training data.

### CROSS-REFERENCE TO RELATED APPLICATION

This application claims priority from Korean Patent Application No. 10-2022-0164218, filed on Nov. 30, 2022, in the Korean Intellectual Property Office, the entire disclosure of which is incorporated herein by reference.

### BACKGROUND

### 1. Field

Example embodiments of the present disclosure relate to an apparatus and method for non-invasively estimating an antioxidant concentration.

### 2. Description of Related Art

Recently, research has been conducted on a method of non-invasively estimating components, such as blood glucose, carotenoid, etc., by reflectance spectrum measurement using Raman Spectroscopy or near-infrared spectroscopy to provide commercial equipment in a smaller size. Generally, substances, such as hemoglobin, carotene, melanin, etc., are determinants of skin color. Particularly, melanin absorbs much light, thereby resulting in performance degradation, such as reduction in signal to noise ratio, of a spectrum-based sensor, and the like. That is, not only the effects of melanin pigment and hemoglobin which are determinants of skin color, but also an effect resulting from measurement position are reflected in an absorbance spectrum, thereby decreasing the accuracy in stably measuring components, such as carotenoid, from people with various characteristics according to various skin colors and measurement positions.

### SUMMARY

One or more example embodiments provide an apparatus and method for non-invasively estimating an antioxidant concentration.

According to an aspect of an example embodiment, there is provided an apparatus configured to estimate an antioxidant component, the apparatus including a storage configured to store first antioxidant concentrations estimated at a first body part and second antioxidant concentrations estimated at a second body part, and a processor configured to estimate the first antioxidant concentrations at the first body part, to extract, as training data, data pairs of the first antioxidant concentrations and the second antioxidant concentrations from the storage based on learning conditions, and generate a transformation model configured to transfer the second antioxidant concentrations into a reference index based on the training data.

The apparatus may further include one or more sensors configured to measure optical signals from body parts of a user.

The processor may be further configured to extract a first antioxidant concentration and a second antioxidant concentration, corresponding to a time at which the first antioxidant concentration is estimated, as data pair among the data pairs from the storage.

The processor may be further configured to extract data pairs of the first antioxidant concentrations and the second antioxidant concentrations from the storage included in a predetermined period based on at least one of a color, a thickness, and a structure of a user's skin and physiological characteristics.

Based on second antioxidant concentration, corresponding to the time at which the first antioxidant concentration is estimated, not being included in the storage, the processor may be further configured to extract data pair of a first antioxidant concentration and a second antioxidant concentration from the storage based on one or more adjacent second antioxidant concentrations estimated at times prior to or after the time at which the first antioxidant concentration is estimated.

Based on a time point at which a change in absorbance of an optical signal measured at the second body part is greater than or equal to a threshold value, the processor may be further configured to extract data pairs of the first antioxidant concentrations and the second antioxidant concentrations from the storage at a time interval after the time point.

The processor may be further configured to extract the data pairs of the training data until a number of data pairs is greater than or equal to a predetermined number.

Based on generating the transformation model, the processor may be further configured to perform update by transforming the second antioxidant concentrations, stored in the storage, into the reference index based on the generated transformation model.

The processor may be further configured to transform second antioxidant concentrations, included in first data pair to last data pair of the training data of the generated transformation model, into the reference index.

Based on a missing interval existing between the first data pair of the training data of the generated transformation model and the last data pair of the training data of a transformation model generated at a previous time, the processor may be further configured to transform second antioxidant concentrations in the missing interval based on at least one of the two transformation models.

The processor may be further configured to obtain, as the reference index, an arithmetic mean or a weighted average of values obtained by transforming the second antioxidant concentrations in the missing interval based on each of the two transformation models, or select one transformation model based on a distance between times at which each of the two transformation models are generated and times at which the second antioxidant concentrations in the missing interval are estimated, and transform the second antioxidant concentrations in the missing interval into the reference index based on the selected transformation model.

The processor may be further configured to transform the second antioxidant concentrations of the data pairs, included in the training data of the generated transformation model and in the training data of the transformation model generated at the previous time, based on the two transformation models.

The processor may be further configured to obtain, as the reference index, an arithmetic mean or a weighted average of values obtained by transforming the second antioxidant concentrations of the data pairs, included in the training data of the generated transformation model and in the training data of the transformation model generated at the previous time, by using the two transformation models, or integrate the two transformation models, and transform the second antioxidant concentrations of the data pairs, included in the training data of the generated transformation model and in the training data of the transformation model generated at the previous time, based on the integrated transformation model.

According to another aspect of an example embodiment, there is provided a method of estimating an antioxidant component, the method including estimating first antioxidant concentrations at a first body part, extracting, as training data, data pairs of the first antioxidant concentrations estimated at the first body part and second antioxidant concentrations estimated at a second body part from the storage based on learning conditions, and generating a transformation model configured to transform the second antioxidant concentrations into a reference index based on the training data.

Configuration of the training data may include extracting a first antioxidant concentration and a second antioxidant concentration, corresponding to a time at which the first antioxidant concentration is estimated, as data pair of the first antioxidant concentration and the second antioxidant concentration from the storage.

The extracting may further include extracting data pairs of the first antioxidant concentrations estimated at the first body part and second antioxidant concentrations estimated at a second body part from the storage included in a predetermined period based on at least one of a color, a thickness, and a structure of a user's skin and physiological characteristics.

The extracting may further include, based on a time point at which a change in absorbance of an optical signal measured at the second body part is greater than or equal to a threshold value, extracting data pairs of the first antioxidant concentrations estimated at the first body part and second antioxidant concentrations estimated at a second body part from the storage at a time interval after the time point.

The extracting may further include extracting the data pairs of the training data until a number of data pairs is greater than or equal to a predetermined number.

The method may further include, based on generating the transformation model, updating by transforming the second antioxidant concentrations stored in the storage, into a reference index based on the generated transformation model.

According to another aspect of an example embodiment, there is provided an apparatus configured to estimate an antioxidant component, the apparatus including a sensor configured to measure an optical signal from a body part of a user at which an antioxidant concentration is to be estimated, a processor, based on the optical signal being measured at the body part, configured to estimate the antioxidant concentration based on the measured optical signal, and to transform the estimated antioxidant concentration into a reference index based on a transformation model, and an output interface configured to output the transformed reference index, wherein the transformation model is a model that is trained to transform the antioxidant concentration, measured at the body part, into the reference index based on training data including one or more data pairs of an antioxidant concentration estimated at a reference part and the antioxidant concentration estimated at the body part.

### BRIEF DESCRIPTION OF THE DRAWINGS

The above and other aspects, features, and advantages of example embodiments of the disclosure will be more apparent from the following description taken in conjunction with the accompanying drawings, in which:

FIG. 1 is a block diagram illustrating an apparatus for estimating an antioxidant component according to an example embodiment:

FIGS. 2A, 2B, and 2C are diagrams illustrating examples of generating transformation models:

FIGS. 3A, 3B, and 3C are diagrams illustrating example of applying transformation models:

FIG. 4 is a block diagram illustrating an apparatus for estimating an antioxidant component according to another example embodiment:

FIG. 5 is a diagram illustrating an example of outputting an antioxidant concentration estimation result:

FIG. 6 is a flowchart illustrating a method of estimating an antioxidant component according to an example embodiment; and

FIGS. 7, 8, and 9 are diagrams illustrating examples of various structures of an electronic device including the aforementioned apparatus for estimating an antioxidant component.

### DETAILED DESCRIPTION

Details of example embodiments are included in the following detailed description and drawings. Advantages and features of the present disclosure, and a method of achieving the same will be more clearly understood from the following example embodiments described in detail with reference to the accompanying drawings. Throughout the drawings and the detailed description, unless otherwise described, the same drawing reference numerals will be understood to refer to the same elements, features, and structures.

It will be understood that, although the terms first, second, etc. may be used herein to describe various elements, these elements should not be limited by these terms. These terms are only used to distinguish one element from another. Any references to singular may include plural unless expressly stated otherwise. In addition, unless explicitly described to the contrary, an expression such as “comprising” or “including” will be understood to imply the inclusion of stated elements but not the exclusion of any other elements. Also, the terms, such as “unit” or “module”, etc., should be understood as a unit for performing at least one function or operation and that may be embodied as hardware, software, or a combination thereof.

Hereinafter, embodiments of an apparatus and method configured to estimate an antioxidant component will be described with reference to the accompanying drawings. The embodiments of an apparatus and method configured to estimate an antioxidant component may be included in various types of wearable devices, such as a smartwatch, a smart band wearable device, a headphone wearable device, a headband wearable device, etc., or mobile devices, such as a smartphone, a tablet PC, etc., or electronic devices in specialized medical institution systems. However, the embodiments thereof are not limited thereto.

Various embodiments of estimating an antioxidant component which will be described below may be used configured to estimate components, such as blood glucose, amount of sugar intake, urea, lactate, triglyceride, protein, calories, cholesterol, ethanol, in vivo water, in vitro water, etc., in addition to the antioxidant component.

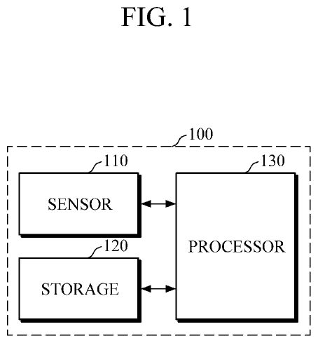

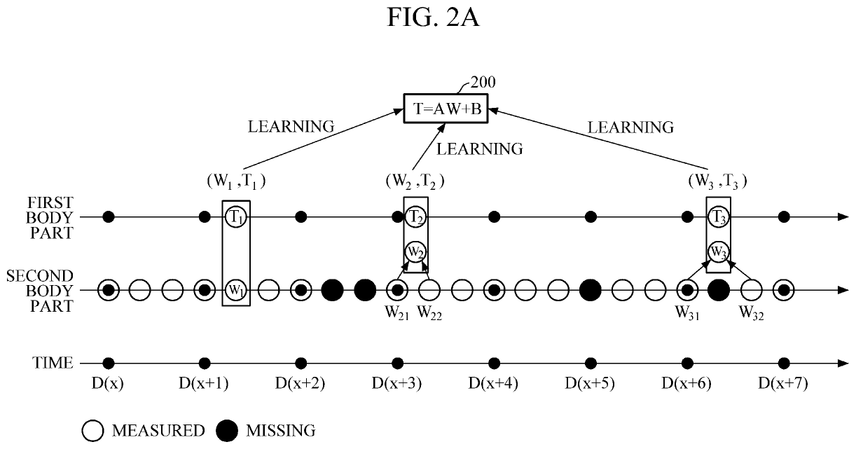

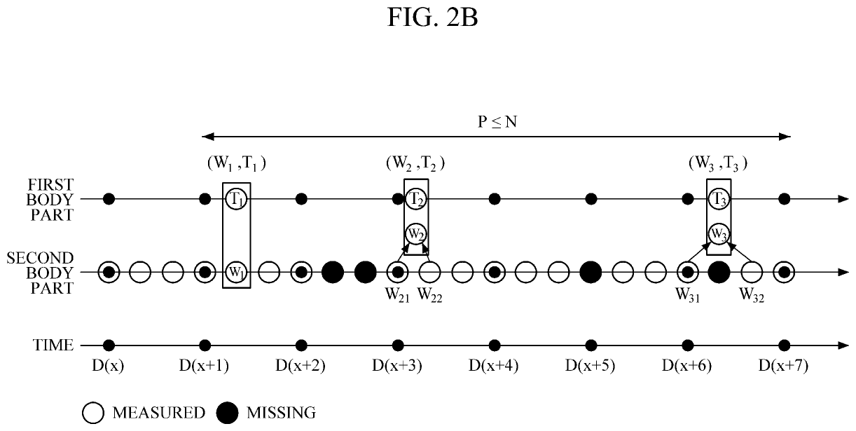

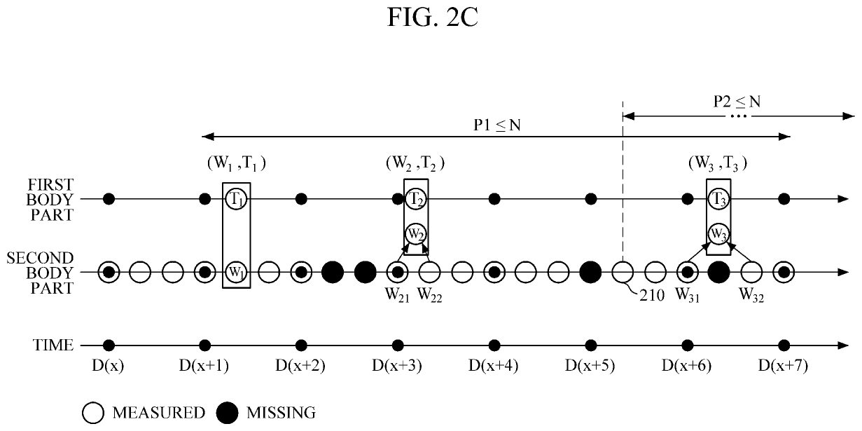

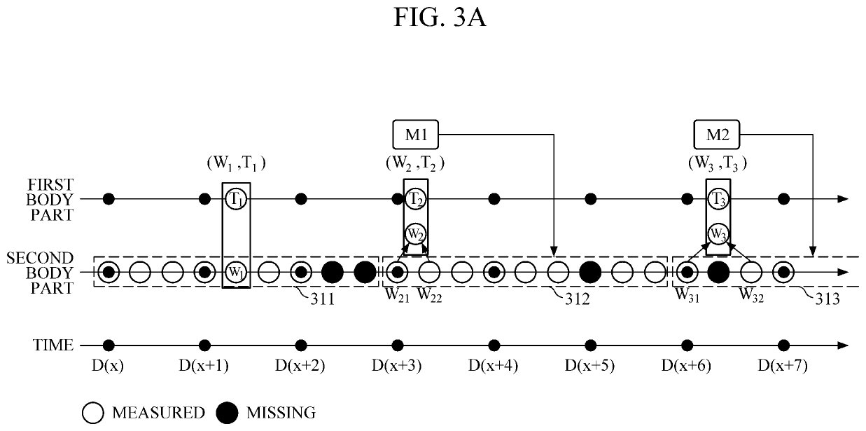

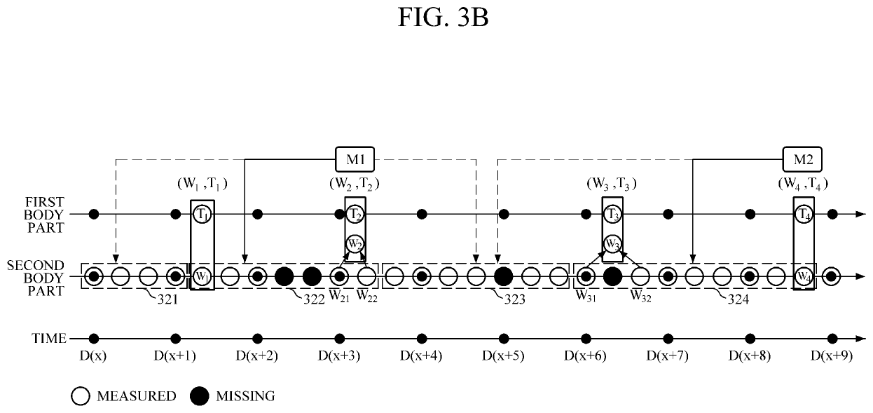

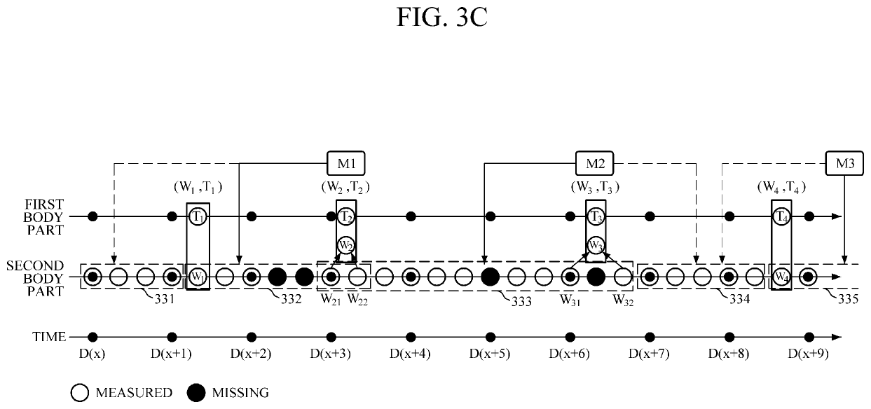

FIG. 1 is a block diagram illustrating an apparatus configured to estimate an antioxidant component according to an example embodiment. FIGS. 2A to 2C are diagrams illustrating examples of generating transformation models. FIGS. 3A to 3C are diagrams illustrating example of applying transformation models.

Referring to FIG. 1, an apparatus 100 configured to estimate an antioxidant component includes a sensor 110, a storage 120, and a processor 130.

The sensor 110 may measure an optical signal from a user's object. In this case, the object may be, for example, the palm of the hand or the sole of the foot which is covered with a relatively thick epidermis layer, a body part that is adjacent to the radial artery or where venous blood or capillary blood is located, a peripheral part of the body with high blood vessel density, such as fingers, toes, earlobes, etc., or a body part, such as the wrist and an inner part of the ear which may come into contact with a wearable device when the device is worn, and the like.

The sensor 110 may include a light source configured to emit light to an object. The light source may be a light emitting diode (LED), a laser diode, and a phosphor. One or a plurality of light sources may be provided, in which case the respective light sources may have different central wavelengths, for example, a central wavelength ranging from 350 nm to 450 nm, a central wavelength ranging from 450 nm to 500 nm, a central wavelength ranging from 500 nm to 550 nm, a central wavelength ranging from 550 nm to 650 nm, and the like. However, the central wavelengths of the light sources are not limited thereto, and the number of light sources, a wavelength range, and the like may vary according to various conditions, such as the type of a component to be estimated, the size of a form factor of the apparatus 100, and the like. The plurality of light sources may be driven simultaneously or sequentially in a time-division manner. According to another example embodiment, the light sources may be driven in a generally sequential manner by simultaneously driving predetermined light sources in groups.

The sensor 110 may include a detector configured to detect light scattered or reflected from or transmitted into the object, to convert the light into an electrical signal, and to output the electrical signal. The detector may include a photo diode, a photo transistor (PTr), or an array thereof, but is not limited thereto, and may include a complementary metal oxide semiconductor (CMOS) image sensor, a charge-coupled device (CCD) image sensor, and the like.

One or more sensors 110 may be provided at a position at which the object may more easily come into contact with the sensors 110. For example, one sensor 110 may be disposed on a front surface, a rear surface, or a side surface of the apparatus 100. According to another example embodiment, in order to simultaneously measure optical signals at two or more body parts, two or more sensors may be disposed at two or more positions, such as the front surface, rear surface, side surface, and the like of the apparatus 100.

In addition, the sensor 110 may include an analog-digital converter configured to convert an electrical signal, output from the detector, into a digital signal and/or an amplifier configured to amplify the electrical signal.

However, the sensor 110 is not limited to the above description, and may be a sensor to which, for example, Raman Spectroscopy, near-infrared spectroscopy, or mid-infrared spectroscopy is applied.

The storage 120 may store a variety of information configured to estimate a concentration of an antioxidant component. For example, when the sensor 110 measures the optical signal, the storage 120 may store the measured optical signal, a body part where the measurement is performed, and/or measurement time data. In addition, when the processor 130 estimates an antioxidant concentration, the storage 120 may store the estimated antioxidant concentration and a time at which the concentration is estimated. Further, when the processor 130 generates a transformation model, the storage 120 may store the generated transformation model, and a time at which the transformation model is generated. In addition, the storage 120 may store color, thickness, and structure of a user's skin, and physiological characteristics such as height, weight, age, disease data, and the like of a user. Furthermore, the storage 120 may store sensor operating condition data including a light source operating condition and the like.

The storage 120 may include at least one storage medium of a flash memory type memory, a hard disk type memory, a multimedia card micro type memory, a card type memory (e.g., an SD memory, an XD memory, etc.), a random access memory (RAM), a static random access memory (SRAM), a read only memory (ROM), an electrically erasable programmable read only memory (EEPROM), a programmable read only memory (PROM), a magnetic memory, a magnetic disk, and an optical disk, and the like, but is not limited thereto. For example, the storage 120 may include a storage medium, such as a cloud storage medium, which is separately provided, in addition to a storage medium which is included in a single hardware along with the sensor 110 and the processor 130.

The processor 130 may be electrically connected to the sensor 110. The processor 130 may control the sensor 110 to obtain an optical signal from a body part where a concentration is to be estimated, and may estimate an antioxidant concentration by using the obtained optical signal. In this case, the body part where the concentration is estimated may be a position where the measurement may be more easily performed when a user wears or carries the apparatus 100, and may be an outer part of the wrist or the back of the hand. The concentration estimation time may include a time requested by a user, and may be set to continuous time intervals or relatively short time intervals (e.g., 15 minutes, one hour, two hours, three times a day, etc.).

When a plurality of optical signals having different wavelengths are obtained, the processor 130 may reconstruct a spectrum based on the obtained optical signals. Upon reconstructing the spectrum, the processor 130 may obtain an absorbance spectrum by using the reconstructed spectrum, and may estimate an antioxidant concentration by applying a predefined concentration estimation model to absorbance at each wavelength. In this case, the concentration estimation model may be defined as, for example, a linear combination equation or a neural network-based model, but is not limited thereto. Based on a calibration spectrum which is obtained based on an initial light quantity measured for each light source by using a standard reflector, the processor 130 may obtain the absorbance spectrum by correcting the reconstructed spectrum. In this case, the standard reflector may be a reflector having a predetermined reflectance, e.g., average reflectance (about 50%) or maximum reflectance (about 80%) of human skin.

When a predetermined time has passed, the processor 130 may transform second antioxidant concentrations estimated at a second body part into a reference index based on first antioxidant concentrations estimated at a first body part which is used as a reference for a user. The predetermined time may be a calibration time, a predetermined cycle, or a time requested by a user, and the like. The first body part may be a portion relatively less affected by the effect of melanin pigment, hair, secretor, etc., and may refer to a body part (e.g., finger, palm of the hand, etc.) at which antioxidant concentrations may be estimated for use as an absolute index for comparison of each individual. Accordingly, the reference index may indicate a concentration corresponding to the first body part which may be used as an absolute index.

When a calibration time has passed, the processor 130 may control the sensor 110 to obtain an optical signal at the first body part, and may estimate a first antioxidant concentration by using the obtained optical signal. The calibration time may be set to a time interval (e.g., 5, 7, 10, or 30 days) which is relatively longer than a concentration estimation time, and may include a time requested by a user.

The sensor 110 that measures the optical signal at the first body part may be the same as the sensor 110 that measures the optical signal at the second body part. In this case, after first measuring the optical signal at one of two body parts, the sensor 110 may successively measure the optical signal at the other body part. In addition, in the case where two or more sensors 110 are provided at different positions, the sensors 110 that are different from each other may measure the optical signals at both the body parts. In this case, when one sensor 110 measures the optical signal at the first body part, the other sensor 110 may measure the optical signal at the second body part.

According to another example embodiment, when another electronic device measures the optical signal at the first body part or estimates an antioxidant concentration at a calibration time, the processor 130 may receive the optical signal or antioxidant concentration data from the electronic device, or the data may be input by a user. The electronic device may be a device, such as a smartphone, a tablet PC, a laptop computer, a desktop computer, etc., or an Internet of things (IOT) device including home appliances, such as a refrigerator, a microwave oven, a washing machine, and the like.

Upon estimating the antioxidant concentrations at the first body part and the second body part, the processor 130 may store the first antioxidant concentration estimated at the first body part and an estimation time at which the first antioxidant concentration is estimated and the second antioxidant concentration estimated at the second body part and an estimation time at which the second antioxidant concentration is estimated in the storage 120.

Upon estimating the first antioxidant concentration at the first body part, the processor 130 may extract, as training data, data according to learning conditions by referring to the storage 120, and may train a basic transformation model by using the training data. Upon determining parameter values by training the basic transformation model, the processor 130 may generate a new transformation model, and may store the generated transformation model and a time at which the transformation model is generated in the storage 120. The transformation model may be a model for transforming second antioxidant concentrations estimated at the second body part into a reference index. The transformation model may be defined as a linear or non-linear function or a neural network-based model. The following Equation 1 is an example of a basic transformation model represented by a simple linear function.

T=AW+B [Equation 1]

Herein, T denotes the transformed reference index, W denotes the second antioxidant concentration before transformation, and A and B denotes parameters of the transformation model and are values determined by training. In this case, some of the parameters (e.g., A indicating a slope) may be an arbitrary constant and may be pre-defined.

Upon estimating the first antioxidant concentration at the first body part and storing the concentration in the storage 120, the processor 130 may extract one or more data pairs of the first antioxidant concentrations and second antioxidant concentrations, corresponding to estimation times of the respective first antioxidant concentrations, as training data from the storage 120. In this case, if there is no second antioxidant concentration corresponding to an estimation time of a first antioxidant concentration, one or more adjacent second antioxidant concentrations estimated at times relatively close to the estimation time of the first antioxidant concentration may be used. For example, the processor 130 may determine an arithmetic mean of the adjacent second antioxidant concentrations on both sides thereof, a weighted average obtained by assigning a higher weight to a second antioxidant concentration estimated at a time, which is closer to the estimation time of the first antioxidant concentration, of the second antioxidant concentrations on both sides thereof, or the closer second antioxidant concentration itself, as the second antioxidant concentration of the training data.

Referring to FIG. 2A, upon estimating a first antioxidant concentration T3 at the first body part at a current time and storing the estimated concentration in the storage 120, the processor 130 may extract, as training data, data pairs (W1,T1), (W2,T2), and (W3,T3) of first antioxidant concentrations T1, T2, and T3 and second antioxidant concentrations W1, W2, and W3 corresponding thereto which are stored in the storage 120, and may train a basic transformation model 200. In this case, the first antioxidant concentrations T2 and T3 may be estimated at different times or may be missing, such that there are no second antioxidant concentrations corresponding thereto, in which case the processor 130 may obtain the second antioxidant concentrations W2 and W3 based on adjacent second antioxidant concentrations (W21,W22) and (W31,W32).

The processor 130 may extract data pairs of training data according to predetermined learning conditions. A first learning condition may be set so that a learning interval P may be within a predetermined period N as illustrated in FIG. 2B. Upon estimating the first antioxidant concentration T3 at the current time, the processor 120 may extract data pairs within the predetermined period N from the current time. In this case, the transformation model is affected by user characteristics, e.g., the color, thickness, structure of skin at a body part, and physiological characteristics and the like, such that the period N may be set to various periods, such as a week, half a month, a month, a quarter, half a year, a year, unlimited, etc., so as not to cause a significant change in user characteristics. The predetermined period N may be adjusted by a user. In addition, the processor 130 may set the period N or may adjust the predetermined period N by further considering environmental factors, such as a time period when a user mainly wears the apparatus 100, a wearing time of the apparatus 100 during the day, season, and the like. For example, if the user does not wear the apparatus 100 during daytime or wears the apparatus 100 for a short period time, or as there is a relatively higher possibility of skin color change during summertime, the processor 130 may set the period N to a shorter period.

A second learning condition may be set to extract data pairs of training data in consideration of a change in absorbance of an optical signal measured at the second body part. Generally, if there is a change in skin condition at a specific time due to shaving, skin trouble, excessive tanning, etc., there may be an excessive change in absorbance of an optical signal measured at that time compared to a previous time. Accordingly, the processor 130 may compare a reference absorbance with absorbance at a specific time which is used for determining an absorbance change, and if the absorbance change (rate of change or variation) is greater than or equal to a threshold value (e.g., 7%, 10%, etc.), the processor 130 may extract data pairs in an interval after the specific time. The threshold value may be set for each user. Referring to FIG. 2C, the processor 130 may extract data pairs of the training data in a learning interval P1, which is within the predetermined period N, according to the aforementioned first learning condition, in which case if there is a time point 210, at which a change in absorbance is greater than a threshold value, in the learning interval P1, the processor 130 may extract data pairs of the training data in a new learning interval P2, which is within the predetermined period N, after the time point 210. In this case, the reference absorbance may be absorbance of an optical signal measured at a calibration time before the specific time point, or absorbance of an optical signal measured at the second body part at a time immediately before the specific time point, or a statistical value (e.g. mean value) of absorbance of optical signals measured at the second body part in the learning interval. If there are absorbance of optical signals in a plurality of wavelength ranges, the processor 130 may compare the reference absorbance with absorbance in a predetermined specific wavelength range or a statistical value (e.g. mean value) of absorbance in the entire wavelength range.

A third learning condition may be set so that the number of data pairs of training data may be a predetermined number or more. For example, the number of data pairs may be set to two or more in consideration of the number of parameters of the transformation model, but if some of the parameters are set to a default value, the number of data pairs may be set to one. If the number of data pairs that satisfy the first and second learning conditions is not greater than or equal to the predetermined number, the processor 130 may not train the transformation model until a next calibration time.

The above learning conditions are merely examples, and in order to prevent the respective data pairs from being used for training two or more transformation models, it may also be set to exclude the data pair, used for training a previous transformation model, from training data of a current transformation model. Two or more of the above learning conditions may be combined, or specific conditions may be omitted.

The processor 130 may determine a parameter by training a basic transformation model using training data that satisfy the learning conditions, and may generate a new transformation model by using the determined parameter. In addition, the processor 130 may store the generated transformation model and a time at which the transformation model is generated in the storage 120. Further, upon generating the new transformation model, the processor 130 may perform updating by transforming the second antioxidant concentrations, stored in the storage 120, into a reference index by using the transformation model.

Referring to FIG. 3A, upon estimating the second antioxidant concentrations at the second body part, the processor 130 may transform the second antioxidant concentrations in real time by using a transformation model generated before the estimation time. According to another example embodiment, a transformation period (e.g., a day, before calibration, etc.) may be set in advance. The processor 130 may transform the second antioxidant concentrations in response to a user's request. As illustrated herein, the processor 130 may transform the second antioxidant concentrations in an interval 312 by using a transformation model M1 generated before the interval. Likewise, the processor 130 may transform the second antioxidant concentrations in an interval 313 by using a transformation model M2 generated before the interval. In this case, in an interval 311 in which no previous transformation model is present, such as in the case where the apparatus 100 is first used or is initialized, the processor 130 may retrospectively transform the second antioxidant concentrations by using the transformation model M1 generated after the interval.

Referring to FIG. 3B, upon generating a transformation model, the processor 130 may retrospectively transform the second antioxidant concentrations included in first to last data pairs of the training data of the transformation model. As illustrated herein, upon generating the transformation model M1, the processor 130 may transform the second antioxidant concentrations in an interval 322 between data pairs (W1,T1) and (W2,T2) of the training data of the transformation model M1. Likewise, upon generating the transformation model M2, the processor 130 may transform the second antioxidant concentrations in an interval 324 between data pairs (W3,T3) and (W4,T4) of the training data by using the transformation model M2.

In this case, the processor 130 may transform second antioxidant concentrations in a missing interval 323, to which the two transformation models M1 and M2 are not applied, for example, an interval between the last data pair of the transformation model M1 and the first data pair of the transformation model M2, by using the two transformation models M1 and M2. For example, the processor 130 may transform the second antioxidant concentrations in the missing interval 323 by using each of the transformation model M1 and the transformation model M2, and may obtain, as a reference index, an arithmetic mean of the transformed concentrations or a weighted average according to a time interval between time points at which the second antioxidant concentrations are estimated and time points at which the two transformation models M1 and M2 are generated. According to another example embodiment, the processor 130 may select one transformation model in an interval close to the estimation time of the second antioxidant concentration, and may transform the second antioxidant concentration by using the selected transformation model. As for a missing interval 321 before the transformation model M1, the processor 130 may transform the second antioxidant concentrations in the interval 321 by using the transformation model M1.

Referring to FIG. 3C, upon generating the transformation models as illustrated in FIG. 3B, the processor 130 may retrospectively transform the second antioxidant concentrations included in the first to last data pairs of the training data of the transformation models. In this case, the processor 130 may transform the second antioxidant concentrations, which are commonly used in training data of two or more transformation models, by applying both the two or more transformation models. As illustrated herein, by transforming the second antioxidant concentrations W21 and W22, which are commonly used in the training data of the two transformation models M1 and M2, an arithmetic mean of the concentrations transformed by using the two transformation models M1 and M2 or a weighted average of times may be obtained as a reference index. According to another example embodiment, the processor 130 may generate an integrated transformation model by integrating the two transformation models M1 and M2, and may transform the second antioxidant concentrations by applying the integrated transformation model. For example, the processor 130 may calculate statistical values (e.g., mean values (A1+A2)/2 and (B1+B2)/2)) of parameters A1 and B1 of the transformation model M1 and parameters A2 and B2 of the transformation model M2 as parameters of the integrated transformation model, and may generate the integrated transformation model by using the calculated parameters.

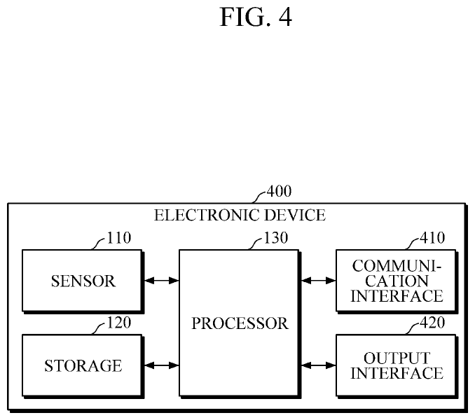

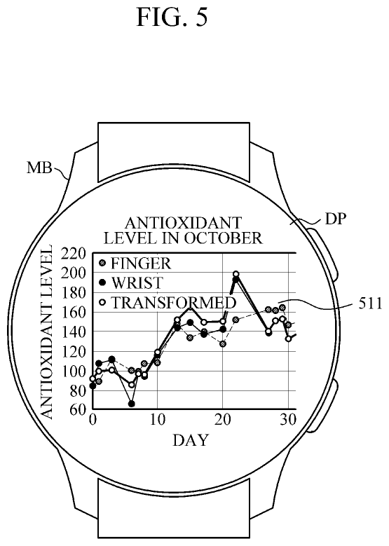

FIG. 4 is a block diagram illustrating an apparatus configured to estimate an antioxidant component according to another example embodiment. FIG. 5 is a diagram illustrating an example of outputting an antioxidant concentration estimation result.

Referring to FIG. 4, an apparatus 400 configured to estimate an antioxidant component includes the sensor 110, the storage 120, the processor 130, a communication interface 410, and an output interface 420. The sensor 110, the storage 120, and the processor 120 are described in detail above, such that the following description will be given of non-overlapping parts.

The communication interface 410 may communicate with another external device by wired or wireless communications to transmit and receive various data related to estimating an antioxidant component. For example, the communication interface 410 may transmit the first antioxidant concentration, the second antioxidant concentration, and the transformed reference index to another electronic device, and may receive the first antioxidant concentration or the optical signal measured at the first body part by another electronic device. The external device may include an information processing device such as a smartphone, a tablet PC, a laptop computer, a desktop computer, etc., but is not limited thereto.

The communication interface 410 may communicate with the external device by using communication techniques, such as Bluetooth communication, Bluetooth Low Energy (BLE) communication, near field communication (NFC), WLAN communication, Zigbee communication, infrared data association (IrDA) communication, Wi-Fi Direct (WFD) communication, ultra-wideband (UWB) communication, Ant+ communication, WIFI communication, radio frequency identification (RFID) communication, 3G, 4G, and 5G communications, and the like. However, this is merely exemplary and is not intended to be limiting.

The output interface 420 may include an output module, such as a display, a speaker, and/or a haptic device using vibrations, tactile sensation, etc., and may visually/non-visually output the optical signals measured at the first body part and the second body part, the first antioxidant concentration, the second antioxidant concentration, the reference index transformed by using the transformation model, and the like. As illustrated in FIG. 5, the output interface 420 may output, to a display DP of a main body MB, antioxidant concentrations measured at a first body part (finger) and a second body part (wrist) over a predetermined period of time and/or a change trend of the transformed reference index in a graph. In addition, when a user selects a graphic object 511 indicating a specific time point in the change trend graph, the output interface 420 may output detailed information about an antioxidant concentration at the corresponding time point, for example, a measurement position at the corresponding time point, a measurement time, an antioxidant concentration, a transformed concentration, and the like. In addition, the output interface 420 may output information for guiding a user to place a first sensor portion or a second sensor portion on the sensor 110 at a calibration time or a measurement time.

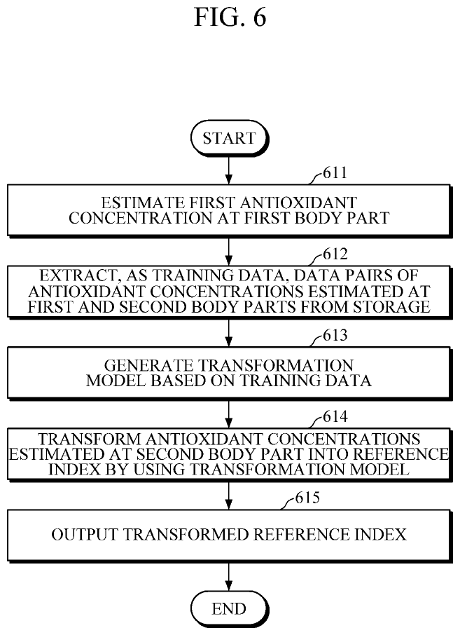

FIG. 6 is a flowchart illustrating a method of estimating an antioxidant component according to an example embodiment.

The method of FIG. 6 is an example of a method of estimating an antioxidant component performed by the apparatus configured to estimate an antioxidant component of FIG. 1 or FIG. 4, and thus will be briefly described below.

First, the apparatus configured to estimate an antioxidant component may estimate a first antioxidant concentration at a first body part of an object in step 611. When a calibration time has arrived, the apparatus configured to estimate an antioxidant component may measure an optical signal at the first body part by using a sensor included in the apparatus configured to estimate an antioxidant component, and may estimate a first antioxidant concentration by using the measured optical signal. According to another example embodiment, the apparatus configured to estimate an antioxidant component may receive an optical signal or a first antioxidant concentration measured at the first body part by another electronic device. Upon estimating the first antioxidant concentration, the apparatus configured to estimate an antioxidant component may store the estimated first antioxidant concentration and an estimation time thereof.

Then, the apparatus configured to estimate an antioxidant component may extract, as training data, data pairs of one or more first antioxidant concentrations and second antioxidant concentrations, corresponding to estimation times of the first antioxidant concentrations, from the storage in step 612. In this case, if there is no second antioxidant concentration corresponding to the estimation time of the first antioxidant concentration, the apparatus configured to estimate an antioxidant component may obtain second antioxidant concentrations by performing interpolation based on one or more adjacent second antioxidant concentrations estimated at times close to the corresponding estimation time. In this case, learning conditions for configuring training data may be preset as described above.

Subsequently, upon configuring the training data, the apparatus configured to estimate an antioxidant component may generate a transformation model based on the training data in step 613. The apparatus configured to estimate an antioxidant component may determine a parameter by training a basic transformation model by using the training data, and may generate a transformation model by using the determined parameter. Upon generating the transformation model, the apparatus configured to estimate an antioxidant component may store the generated transformation model and a time at which the transformation model is generated in the storage.

Next, upon generating the transformation model, the apparatus configured to estimate an antioxidant component may transform the second antioxidant concentrations into a reference index by using the generated transformation model in step 614.

Then, the apparatus configured to estimate an antioxidant component may output the transformed reference index in step 615. As illustrated in FIG. 5, the apparatus configured to estimate an antioxidant component may visually output the first antioxidant concentrations, the second antioxidant concentrations before transformation, and/or the transformed reference index.

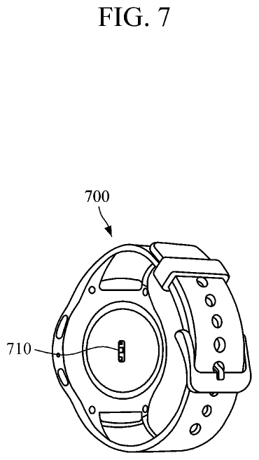

FIGS. 7 to 9 are diagrams illustrating examples of various structures of an electronic device including the aforementioned apparatus configured to estimate an antioxidant component.

The electronic device may include, for example, various types of wearable devices, e.g., a smart watch, a smart band, smart glasses, smart earphones, a smart ring, a smart patch, and a smart necklace, and a mobile device such as a smartphone, a tablet PC, etc., or home appliances or various Internet of Things (IOT) devices (e.g., home IoT device, etc.) based IoT technology.

The electronic device may include a sensor device, a processor, an input device, a communication module, a camera module, an output device, a storage device, and a power module. All the components of the electronic device may be integrally mounted in a specific device or may be distributed in two or more devices. The sensor device may include the sensor 110 of the apparatuses 100 and 400 configured to estimate an antioxidant component, and may further include an additional sensor, such as a gyro sensor, a global positioning system (GPS), and the like.

The processor may execute programs, stored in the storage device, to control components connected to the processor, and may perform various data processing or computation, including estimation of bio-information. The processor may include a main processor, e.g., a central processing unit (CPU) or an application processor (AP), etc., and an auxiliary processor, e.g., a graphics processing unit (GPU), an image signal processor (ISP), a sensor hub processor, or a communication processor (CP), etc., which is operable independently from, or in conjunction with, the main processor.

The input device may receive a command and/or data to be used by each component of the electronic device, from a user and the like. The input device may include, for example, a microphone, a mouse, a keyboard, or a digital pen (e.g., a stylus pen, etc.).

The communication module may support establishment of a direct (e.g., wired) communication channel and/or a wireless communication channel between the electronic device and other electronic device, a server, or the sensor device within a network environment, and performing of communication via the established communication channel. The communication module may include one or more communication processors that are operable independently from the processor and supports a direct communication and/or a wireless communication. The communication module may include a wireless communication module, e.g., a cellular communication module, a short-range wireless communication module, or a global navigation satellite system (GNSS) communication module, etc., and/or a wired communication module, e.g., a local area network (LAN) communication module, a power line communication (PLC) module, and the like. These various types of communication modules may be integrated into a single chip, or may be separately implemented as multiple chips. The wireless communication module may identify and authenticate the electronic device in a communication network by using subscriber information (e.g., international mobile subscriber identity (IMSI), etc.) stored in a subscriber identification module.

The camera module may capture still images or moving images. The camera module may include a lens assembly having one more lenses, image sensors, image signal processors, and/or flashes. The lens assembly included in the camera module may collect light emanating from a subject to be imaged.

The output device may visually/non-visually output data generated or processed by the electronic device. The output device may include a sound output device, a display device, an audio module, and/or a haptic module.

The sound output device may output sound signals to the outside of the electronic device. The sound output device may include a speaker and/or a receiver. The speaker may be used for general purposes, such as playing multimedia or playing record, and the receiver may be used for incoming calls. The receiver may be implemented separately from, or as part of, the speaker.

The display device may visually provide information to the outside of the electronic device. The display device may include, for example, a display, a hologram device, or a projector and control circuitry to control the devices. The display device may include touch circuitry adapted to detect a touch, and/or sensor circuitry (e.g., pressure sensor, etc.) adapted to measure the intensity of force incurred by the touch.

The audio module may convert a sound into an electrical signal or vice versa. The audio module may obtain the sound via the input device, or may output the sound via the sound output device, and/or a speaker and/or a headphone of another electronic device directly or wirelessly connected to the electronic device.

The haptic module may convert an electrical signal into a mechanical stimulus (e.g., vibration, motion, etc.) or electrical stimulus which may be recognized by a user by tactile sensation or kinesthetic sensation. The haptic module may include, for example, a motor, a piezoelectric element, and/or an electric stimulator.

The storage device may store operating conditions required for operating the sensor device, and various data necessary for other components of the electronic device. The various data may include, for example, input data and/or output data for software and instructions associated with the software, and the like. The storage device may include a volatile memory and/or a non-volatile memory.

The power module may manage power supplied to the electronic device. The power module may be implemented as part of, for example, a power management integrated circuit (PMIC). The power module may include a battery, which may include a primary cell which is not rechargeable, a secondary cell which is rechargeable, and/or a fuel cell.

Referring to FIG. 7, the electronic device may be implemented as a wristwatch wearable device 700, and may include a main body and a wrist strap. A display is provided on a front surface of the main body, and may display various application screens, including an estimated concentration value, warning information, time information, received message information, and the like. A sensor device 710 may be disposed on a rear surface of the main body. While being worn on a user's wrist, the wearable device 700 may estimate antioxidant concentrations continuously or at predetermined time intervals. Further, when a calibration time has passed, the wearable device 700 may output, to a display, guide information for guiding a user to estimate an antioxidant concentration at a reference body part, such as a finger and the like. Upon estimating the antioxidant concentration at the reference body part, the wearable device 700 may generate a transformation model by configuring training data, and may transform the antioxidant concentration, measured at the wrist, into a reference index, which is described in detail above, such that a detailed description thereof will be omitted.

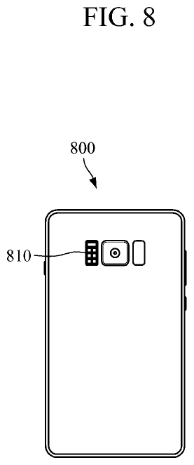

Referring to FIG. 8, the electronic device may be implemented as a mobile device 800 such as a smartphone. The mobile device 800 may include a housing and a display panel. The housing may form an exterior of the mobile device 800. The housing has a first surface, on which a display panel and a cover glass may be disposed sequentially, and the display panel may be exposed to the outside through the cover glass. A sensor device 810, a camera module and/or an infrared sensor, and the like may be disposed on a second surface of the housing. The processor and various other components may be disposed in the housing.

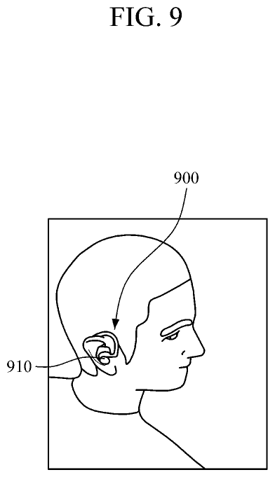

Referring to FIG. 9, the electronic device may be implemented as an ear-wearable device 900. The ear-wearable device 900 may include a main body and an ear strap. A user may wear the ear-wearable device 900 by hanging the ear strap on the auricle. The ear strap may be omitted depending on a shape of the ear-wearable device 900. The main body may be inserted into the external auditory meatus. A sensor device 910 may be mounted in the main body at a portion coming into contact with skin. Further, a processor, a communication device, and the like may be mounted in the main body. By interworking with the wristwatch wearable device 700, the mobile device 800, etc., the ear-wearable device 900 may estimate an antioxidant concentration and/or generate a transformation model, and may transform an antioxidant concentration, estimated at the ear, into a reference index corresponding to a reference body part, such as a finger and the like.

Embodiments of the present disclosure can be realized as a computer-readable code written on a computer-readable recording medium. The computer-readable recording medium may be any type of recording device in which data is stored in a computer-readable manner.

Examples of the computer-readable recording medium include a ROM, a RAM, a CD-ROM, a magnetic tape, a floppy disc, an optical data storage, and a carrier wave (e.g., data transmission through the Internet). The computer-readable recording medium can be distributed over a plurality of computer systems connected to a network so that a computer-readable code is written thereto and executed therefrom in a decentralized manner. Functional programs, codes, and code segments needed for realizing embodiments of the present disclosure can be readily deduced by programmers of ordinary skill in the art.

While example embodiments have been described with reference to the figures, it will be understood by those of ordinary skill in the art that various changes in form and details may be made therein without departing from the spirit and scope as defined by the following claims and their equivalents.

What is claimed is:

1. An apparatus configured to estimate an antioxidant component, the apparatus comprising:
a storage configured to store first antioxidant concentrations estimated at a first body part and second antioxidant concentrations estimated at a second body part; and
a processor configured to:
estimate the first antioxidant concentrations at the first body part, to extract, as training data, data pairs of the first antioxidant concentrations and the second antioxidant concentrations from the storage based on learning conditions; and
generate a transformation model configured to transfer the second antioxidant concentrations into a reference index based on the training data.

2. The apparatus of claim 1, further comprising one or more sensors configured to measure optical signals from body parts of a user.

3. The apparatus of claim 1, wherein the processor is further configured to extract a first antioxidant concentration and a second antioxidant concentration, corresponding to a time at which the first antioxidant concentration is estimated, as data pair among the data pairs from the storage.

4. The apparatus of claim 3, wherein the processor is further configured to extract data pairs of the first antioxidant concentrations and the second antioxidant concentrations from the storage included in a predetermined period based on at least one of a color, a thickness, and a structure of a user's skin and physiological characteristics.

5. The apparatus of claim 3, wherein based on second antioxidant concentration, corresponding to the time at which the first antioxidant concentration is estimated, not being included in the storage, the processor is further configured to extract data pair of a first antioxidant concentration and a second antioxidant concentration from the storage based on one or more adjacent second antioxidant concentrations estimated at times prior to or after the time at which the first antioxidant concentration is estimated.

6. The apparatus of claim 3, wherein based on a time point at which a change in absorbance of an optical signal measured at the second body part is greater than or equal to a threshold value, the processor is further configured to extract data pairs of the first antioxidant concentrations and the second antioxidant concentrations from the storage at a time interval after the time point.

7. The apparatus of claim 1, wherein the processor is further configured to extract the data pairs of the training data until a number of data pairs is greater than or equal to a predetermined number.

8. The apparatus of claim 1, wherein based on generating the transformation model, the processor is further configured to perform update by transforming the second antioxidant concentrations, stored in the storage, into the reference index based on the generated transformation model.

9. The apparatus of claim 8, wherein the processor is further configured to transform second antioxidant concentrations, included in first data pair to last data pair of the training data of the generated transformation model, into the reference index.

10. The apparatus of claim 9, wherein based on a missing interval existing between the first data pair of the training data of the generated transformation model and the last data pair of the training data of a transformation model generated at a previous time, the processor is further configured to transform second antioxidant concentrations in the missing interval based on at least one of the two transformation models.

11. The apparatus of claim 10, wherein the processor is further configured to:
obtain, as the reference index, an arithmetic mean or a weighted average of values obtained by transforming the second antioxidant concentrations in the missing interval based on each of the two transformation models; or
select one transformation model based on a distance between times at which each of the two transformation models are generated and times at which the second antioxidant concentrations in the missing interval are estimated, and transform the second antioxidant concentrations in the missing interval into the reference index based on the selected transformation model.

12. The apparatus of claim 8, wherein the processor is further configured to transform the second antioxidant concentrations of the data pairs, included in the training data of the generated transformation model and in the training data of the transformation model generated at the previous time, based on the two transformation models.

13. The apparatus of claim 12, wherein the processor is further configured to:
obtain, as the reference index, an arithmetic mean or a weighted average of values obtained by transforming the second antioxidant concentrations of the data pairs, included in the training data of the generated transformation model and in the training data of the transformation model generated at the previous time, by using the two transformation models; or
integrate the two transformation models, and transform the second antioxidant concentrations of the data pairs, included in the training data of the generated transformation model and in the training data of the transformation model generated at the previous time, based on the integrated transformation model.

14. A method of estimating an antioxidant component, the method comprising:
estimating first antioxidant concentrations at a first body part;
extracting, as training data, data pairs of the first antioxidant concentrations estimated at the first body part and second antioxidant concentrations estimated at a second body part from the storage based on learning conditions; and
generating a transformation model configured to transform the second antioxidant concentrations into a reference index based on the training data.

15. The method of claim 14, wherein configuration of the training data comprises extracting a first antioxidant concentration and a second antioxidant concentration, corresponding to a time at which the first antioxidant concentration is estimated, as data pair of the first antioxidant concentration and the second antioxidant concentration from the storage.

16. The method of claim 15, wherein the extracting further comprises extracting data pairs of the first antioxidant concentrations estimated at the first body part and second antioxidant concentrations estimated at the second body part from the storage included in a predetermined period based on at least one of a color, a thickness, and a structure of a user's skin and physiological characteristics.

17. The method of claim 15, wherein the extracting further comprises, based on a time point at which a change in absorbance of an optical signal measured at the second body part is greater than or equal to a threshold value, extracting data pairs of the first antioxidant concentrations estimated at the first body part and second antioxidant concentrations estimated at the second body part from the storage at a time interval after the time point.

18. The method of claim 14, wherein the extracting further comprises extracting the data pairs of the training data until a number of data pairs is greater than or equal to a predetermined number.

19. The method of claim 14, further comprising, based on generating the transformation model, performing updating by transforming the second antioxidant concentrations stored in the storage, into a reference index based on the generated transformation model.

20. An apparatus configured to estimate an antioxidant component, the apparatus comprising:
a sensor configured to measure an optical signal from a body part of a user at which an antioxidant concentration is to be estimated;
a processor, based on the optical signal being measured at the body part, configured to estimate the antioxidant concentration based on the measured optical signal, and to transform the estimated antioxidant concentration into a reference index based on a transformation model; and
an output interface configured to output the transformed reference index,
wherein the transformation model is a model that is trained to transform the antioxidant concentration, measured at the body part, into the reference index based on training data comprising one or more data pairs of an antioxidant concentration estimated at a reference part and the antioxidant concentration estimated at the body part.
